(19) United States
(12) Patent Application Publication
(10) Pub. No.: US 2026/0000729 A1
(43) Pub. Date: Jan. 1, 2026

(54) COMBINATION THERAPIES FOR TREATMENT OF CANCER

(71) Applicant: The Regents of the University of Michigan, Ann Arbor, MI (US)

(72) Inventors: Evan T. Keller, Ann Arbor, MI (US); Johann Zimmermann, Müllheim (DE)

(21) Appl. No.: 18/866,645

(22) PCT Filed: May 17, 2023
(86) PCT No.: PCT/US2023/022515

§ 371 Date: Nov. 18, 2024

Related U.S. Application Data
(60) Provisional application No. 63/343,228, filed on May 18, 2022.

(51) Int. Cl. A61K 38/12; A61K 31/337; A61P 35/04

(52) U.S. Cl. CPC A61K 38/12; A61K 31/337; A61P 35/04

(57) ABSTRACT

The present disclosure relates to combination therapies for the treatment of cancer. In particular, the disclosure relates to combination therapy with a CXCR4 inhibitor and a taxane for the treatment of cancer. In some aspects, the disclosure relates to combination therapy with a CXCR4 inhibitor and a taxane for the treatment of tumors in the bone of a subject.

### STATEMENT REGARDING RELATED APPLICATIONS

This application claims priority to U.S. Provisional Patent Application No. 63/343,228, filed May 18, 2022, the entire contents of which are incorporated herein by reference for all purposes.

### FIELD

The present disclosure relates to combination therapies for the treatment of cancer. In particular, the disclosure relates to combination therapy with a CXCR4 inhibitor and a taxane for the treatment of cancer, such as prostate cancer with bone metastasis.

### BACKGROUND

Bone metastasis can occur in any type of cancer, leading to bone pain, skeletal fractures, and increased mortality rates. In particular, prostate cancer is frequently affiliated with bone metastasis. Bone provides a unique and conductive microenvironment for cancer cells to colonize and thrive, and cancer in the bone can therefore be particularly challenging to treat in a subject. Accordingly, what is needed are improved methods for treating bone tumors, in particular those caused by metastasis of prostate cancer in a subject.

### SUMMARY

In some aspects, provided herein are methods of treating cancer in a subject. In some embodiments, provided herein are methods of treating bone cancer or cancer which has metastasized to the bone of a subject. The method comprises providing to the subject a CXCR4 inhibitor and a taxane. In some embodiments, the bone cancer is osteosarcoma, chondrosarcoma, or Ewing sarcoma. In some embodiments, the cancer which has metastasized to the bone of the subject is selected from the group consisting of kidney cancer, lung cancer, lymphoma, multiple myeloma, prostate cancer, and thyroid cancer. In some embodiments, the cancer which has metastasized to the bone of the subject is prostate cancer.

In some embodiments, the CXCR4 inhibitor is a peptide. For example, in some embodiments the CXCR4 inhibitor is Balixafortide. In some embodiments, the taxanc is selected from the group consisting of paclitaxel, docetaxel, abraxane, taxotere, and cabazitaxel.

In some aspects, provided herein is a method of treating a tumor in a bone of a subject, comprising providing to the subject a CXCR4 inhibitor and a taxane. In some embodiments, the CXCR4 inhibitor is a peptide. For example, in some embodiments the CXCR4 inhibitor is Balixafortide. In some embodiments, the taxane is paclitaxel, docetaxel, abraxane, taxotere, or cabazitaxel. In some embodiments, the tumor is the result of bone metastasis of a cancer selected from the group consisting of kidney cancer, lung cancer, lymphoma, multiple myeloma, prostate cancer, and thyroid cancer. For example, in some embodiments the tumor is the result of bone metastasis of prostate cancer.

In some aspects, provided herein are compositions comprising a CXCR4 inhibitor and a taxane for use in a method of treating cancer in a subject, wherein the cancer is a bone cancer or wherein the cancer has metastasized to the bone of a subject. In some embodiments, the CXCR4 inhibitor is a peptide. In some embodiments, the CXCR4 inhibitor is Balixafortide. In some embodiments, the taxane is paclitaxel, docetaxel, abraxane, taxotere, or cabazitaxel. In some embodiments, the bone cancer is osteosarcoma, chondrosarcoma, or Ewing sarcoma. In some embodiments, the cancer which has metastasized to the bone of the subject is selected from the group consisting of kidney cancer, lung cancer, lymphoma, multiple myeloma, prostate cancer, and thyroid cancer.

### DESCRIPTION OF THE DRAWINGS

FIG. 1 shows efficacy of Docetaxel, Balixafortide, and a combination thereof in reducing tumor growth over time. Mice were injected intratibially with PC3-luc cells (2×10⁴ cells in 20 ul) and treated with the designated agent. Tumor burden was measured 1 week, 2 weeks, 3 weeks, and 4 weeks later.

FIG. 2 shows radiograph images of all 59 injected tibia lined up in group (from top down: Vehicle, Docetaxel, Balixafortide, Combo).

FIG. 3A is an example showing how osteolytic area was determined. FIG. 3B is a bar graph quantifying osteolytic areas by treatment.

FIG. 4 shows TRAcP levels of sera collected at time of necropsy. Docetaxel value removed is over three standard deviations away from the mean. Data is shown with the respective means+/−SEM.

FIG. 5A shows ELISA measurements of IL-2 and FIG. 5B shows ELISA measurements of IFN-gamma. Many of the serums were below the lower threshold of the assays.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “co-administration” and variations thereof refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. In some embodiments, when agents or therapies are co-administered, the respective agents or therapies are administered at lower dosages than appropriate for their administration alone. Accordingly, co-administration may be especially desirable in embodiments where the co-administration of two or more agents results in sensitization of a subject to beneficial effects of one of the agents via co-administration of the other agent.

The term “carrier” as used herein refers to any pharmaceutically acceptable solvent of agents that will allow a therapeutic composition to be administered to the subject. A “carrier” as used herein, therefore, refers to such solvent as, but not limited to, water, saline, physiological saline, oil-water emulsions, gels, or any other solvent or combination of solvents and compounds known to one of skill in the art that is pharmaceutically and physiologically acceptable to the recipient human or animal. The term “pharmaceutically acceptable” as used herein refers to a compound or composition that will not impair the physiology of the recipient human or animal to the extent that the viability of the recipient is compromised. For example, “pharmaceutically acceptable” may refer to a compound or composition that does not substantially produce adverse reactions, e.g., toxic, allergic, or immunological reactions, when administered to a subject.

As used herein, the terms “subject” and “patient” are used interchangeably herein and refer to both human and nonhuman animals. The term “nonhuman animals” includes all vertebrates, e.g., mammals and non-mammals, such as nonhuman primates, sheep, dogs, cats, horses, cows, chickens, amphibians, reptiles, and the like. In some embodiments, the subject is a human. In some embodiments, the subject is a male. In some embodiments, the subject is a female. In some embodiments, the subject is suffering from cancer.

As used herein, “treat”, “treating”, “treatment”, and variations thereof refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition. In some embodiments, treating a cancer refers to the management and care of the subject for combating and reducing one or more symptoms of the cancer. For example, treating cancer may reduce tumor burden (e.g. reduce the size of one or more tumors in the subject afflicted with cancer and/or reduce the overall number of tumors in the subject afflicted with cancer). Treating a cancer may reduce or completely eliminate the cancer (e.g. completely eliminate the tumor) in the subject.

### DETAILED DESCRIPTION

In some aspects, provided herein are methods of treating cancer in a subject. In some embodiments, methods of treating cancer in a subject comprise providing to the subject a composition described herein. In some embodiments, the methods comprise providing to the subject a C—X—C chemokine receptor type 4 (CXCR4) inhibitor and a taxane. CXCR4, also known as “fusin”, is a G-protein coupled receptor that was initially discovered for its involvement in HIB entry and leukocyte trafficking. CXCR4 has been found to be overexpressed in more than 23 cancer types in humans. Overexpression of CXCR4 in cancer cells contributes to tumor growth, invasion, angiogenesis, metastasis, relapse, and therapeutic resistance.

Any suitable CXCR4 inhibitor may be used. Suitable inhibitors include, for example, antibodies, antibody fragments, aptamers, peptides, and small molecules. In some embodiments, the CXCR4 inhibitor is a peptide. In some embodiments, the CXCR4 inhibitor is the peptide Balixafortide (POL6326). In some embodiments, the CXCR4 inhibitor is Plerixafor (AMD3100). Plerixaflor is an azamacrocycle consisting of two cyclam rings connected by a 1,4-phenylenebis(methylene) linker. The formula of Plerixaflor is C₂₈H₅₄N₈. The chemical name for Plerixaflor is 1-[[4-(1,4,8,11-tetrazacyclotetradec-1-ylmethyl)phenyl]methyl]-1,4,8,11-tetrazacyclotetradecane and it has the structure:

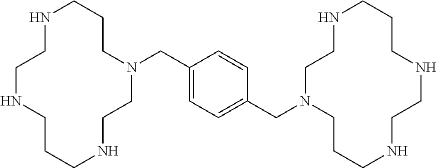 [SMILES: c1cc(CN2CCCNCCNCCCNCC2)ccc1CN1CCCNCCNCCCNCC1]

In some embodiments, the CXCR4 antagonist is Motixafortide (formerly known as BL-8040 or BKT140). Motixafortide is a heterodetic cyclic peptide with the formula C₉₇H₁₄₄FN₃₃O₁₉S₂. The chemical name is (3S,6S,9S,12S,15R,20R,23S,26S,29S,32S)-3,6-bis(4-aminobutyl)-N-[(2S)-1-amino-5-carbamimidamido-1-oxopentan-2-yl]-15-[[(2S)-2-[[(2S)-5-carbamimidamido-2-[[(2S)-5-carbamimidamido-2-[(4-fluorobenzoyl)amino]pentanoyl]amino]pontanoyl]amino]-3-naphthalen-2-ylpropanoyl]amino]-26-(3-carbamimidamidopropyl)-9,23-bis[3-(carbamoylamino)propyl]-12,29-bis[(4-hydroxyphenyl)methyl]-2,5,8,11,14,22,25,28,31-nonaoxo-17,18-dithia-1,4,7,10,13,21,24,27,30-nonazabicyclo[30.3.0]pentatriacontane-20-carboxamide, and the structure of Motixafortide is:

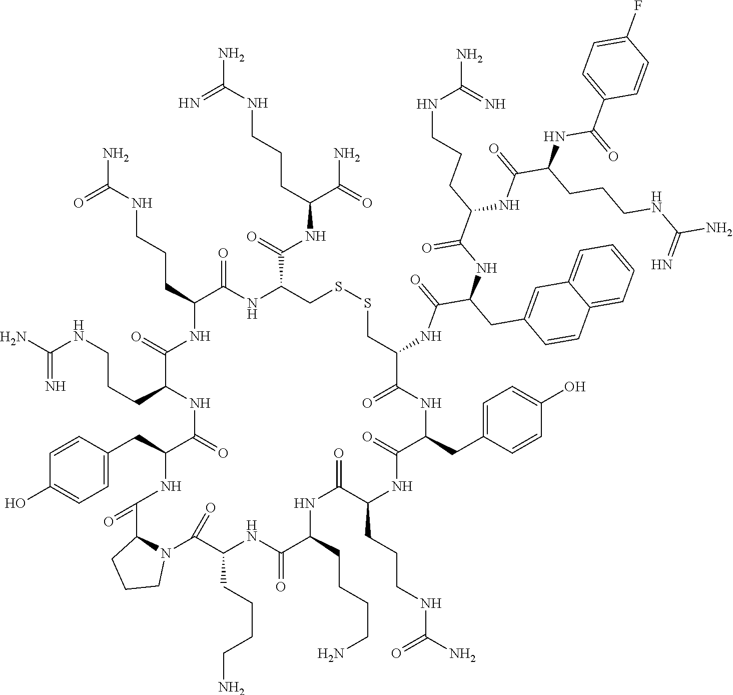 [SMILES: N=C(N)NCCC[C@H](NC(=O)[C@@H]1CSSC[C@H](NC(=O)[C@H](Cc2ccc3ccccc3c2)NC(=O)[C@H](CCCNC(=N)N)NC(=O)[C@H](CCCNC(=N)N)NC(=O)c2ccc(F)cc2)C(=O)N[C@@H](Cc2ccc(O)cc2)C(=O)N[C@@H](CCCNC(N)=O)C(=O)N[C@@H](CCCCN)C(=O)N[C@H](CCCCN)C(=O)N2CCC[C@H]2C(=O)N[C@@H](Cc2ccc(O)cc2)C(=O)N[C@@H](CCCNC(=N)N)C(=O)N[C@@H](CCCNC(N)=O)C(=O)N1)C(N)=O]

In some embodiments, the CXCR4 inhibitor is Balixafortide or an analogue thereof. Balixafortide and analogues thereof are described in U.S. Pat. No. 8,399,611, the entire contents of which are incorporated herein by reference. In some embodiments, the CXCR4 inhibitor may be a Cyclo(-Tyr-His-X-Cys-Ser-Ala-^{D}Pro-Dab-Arg-Tyr-Cys-Tyr-Gln-Lys-^{D}Pro-Pro) compound having a disulfide bond between Cys4 and Cys11. In some embodiments, X is Alanine. Such a compound, cyclo(-Tyr-His-Ala-Cys-Ser-Ala-^{D}Pro-Dab-Arg-Tyr-Cys-Tyr-Gln-Lys-^{D}Pro-Pro-) having a disulfide bond between Cys4 and Cys11, is referred to as Balixafortide. In some embodiments, X is tyrosine. For example, the CXCR4 inhibitor may be Cyclo(-Tyr-His-Tyr-Cys-Ser-Ala-^{D}Pro-Dab-Arg-Tyr-Cys-Tyr-Gln-Lys-” Pro-Pro) compound having a disulfide bond between Cys4 and Cys11.

A taxane is a type of anticancer drug that disrupt mitosis by interfering with microtubules. Suitable taxanes include, for example, paclitaxel, docetaxel, abraxane, taxotere, cabazitaxel, or analogues thereof. The term “analogue” or “analog” are used synonymously herein to refer to a compound having a similar structure to another compound (i.e. a “structural” analogue) and/or to a compound having one or more similar physical, chemical, biochemical, or pharmacological properties to another compound (i.e. a “functional” analogue). For example, a structural analogue may have a similar structure to that of another compound, but differ in one or more atoms, functional groups, or substructures compared to the compound.

In some embodiments, the taxane is selected from the group consisting of paclitaxel, docetaxel, cabazitaxel, larotaxel, ortataxel, tesetaxel, milataxel, docosahexaenoic acid (DHA)-paclitaxel, poly(L-glutamic acid) PG-paclitaxel, BMS-184476 (7-O-methylthiomethyl paclitaxel), SB-T-1214, SB-T-1216, SB-T-121602, SB-T-12854, DHA-SB-T-1214, abeo-taxane 15a.2, cabazitaxel-7,10-d6, docetaxel-f3-t-Boc, docetaxel-d9-t-Boc, ANG-1005, cobalamin-paclitaxel, FK506-PEG3-docetaxel, biotin-docetaxel (IDD-1010), biotin-SB-T-1214 (BLT-1), 4-ARM-PEG20K-CM-Gly-d9-DOC and 4-ARM-PEG20K-BA-d9-DOC, liposomal paclitaxel, Nab™-paclitaxel, Genexol™ PM, and Taclantis™. In some embodiments, the taxane is selected from the group consisting of paclitaxel, docetaxel, abraxane, taxotere and cabazitaxel.

In some embodiments, the taxane is docetaxel. Docetaxel (CAS 114977-28-5) is an antineoplastic agent belonging to the taxoid family which was identified in 1986 as an alternative to paclitaxel. Docetaxel may be prepared by a semi-synthetic process beginning with a precursor extracted from the needles of yew plants (Taxus baccata). The chemical name for docetaxel is (2R,3S)—N-carboxy-3-phenylisoserine,N-tert-butylester, 13 ester with 5ß-20-epoxy-1,2a,4,78,10B,13a-hexahydroxytax-11-en-9-one 4-acetate 2 benzoate, and it has the following chemical structure:

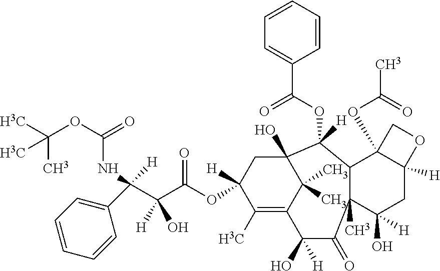

Docetaxel is a white to almost white powder with an empirical formula of C₄₃H₅₃NO₁₄. Docetaxel is described in detail in U.S. Pat. No. 4,814,470, the entire contents of which are incorporated herein by reference.

Other analogues of docetaxel are described in U.S. Patent Publication No. US20060188566A1, the entire contents of which are incorporated herein by reference for all purposes. Such analogues include, for example, 3′-dephenyl-3′cyclohexyldocetaxel, 2-(hexahydro) docetaxel, and 3′-dephenyl-3′cyclohexyl-2-(hexahydro) docetaxel. These docetaxel analogues contain cyclohexyl groups instead of phenyl groups at the C-3′ and/or C-2 benzoate positions. Additional docetaxel analogues include various 2-amido docetaxel analogues, including m-methoxy and m-chlorobenzoylamido analogues, docetaxel analogues lacking the oxetane D-ring but possessing the 4-alpha-acetoxy group, 5 (20) deoxydocetaxel, 10-deoxy-10-C-morpholinoethyl docetaxel analogues, docetaxel analogues having a t-butyl carbamate as the isoserine N-acyl substituent, but differing from docetaxel at C-10 (acetyl group versus hydroxyl) and at the C-13 isoserine linkage (enol ester versus ester), and docetaxel analogues having a peptide side chain at C3.

Additional suitable docetaxel analogues may include XRP9881 (also referred to as RPR 109881A) (10-deacetyl baccatin III docetaxel analogue) (Aventis Pharma), XRP6528 (10-deacetyl baccatin III docetaxel analogue) (Aventis Pharma), Ortataxel (14-beta-hydroxy-deacetyl baccatin III docetaxel analogue) (Bayer/Indena), MAC-321 (10-deacetyl-7-propanoyl baccatin docetaxel analogue) (Wyeth-Ayerst), and DJ-927 (7-deoxy-9-beta-dihydro-9, 10, 0-acetal taxane docetaxal analogue) (Daiichi Pharmaceuticals).

Additional suitable docetaxel analogues include fluorinated docetaxel analogues. Suitable fluorinated analogues include analogues containing one, two or three fluorinc atom(s) either at both meta position on C-2 benzolate and 3′-N-tert-butyloxyl group or only at 3′-N-tert-butyloxyl group, as described in Lu et al., European Journal of Medicinal Chemistry, 44 (2) pp. 482-491, the entire contents of which are incorporated herein by reference.

Additional suitable docetaxel analogues include C-3′ modified analogues. For example, docetaxel analogues may comprise isoxazolyl groups at the C-3′ position. Such analogues are described in Chen et al., Medicinal Chemistry Research 27, 1355-1365 (2018), the entire contents of which are incorporated herein by reference. In some embodiments, docetaxel analogues may additionally comprise a C-3′ modified analogue wherein the C-3′ phenyl group is replaced with a propargyl alcohol. Such compounds are described in Ma et al., J. Nat. Prod, 2018 81 (3) 524-533, the entire contents of which are incorporated herein by reference.

In some embodiments, the taxane is paclitaxel. Paclitaxel (Taxol™), a diterpenoid natural product, is described in Ojima et al., Expert Opin. Ther. Patents 2016, 26 (1), 1-20, and B. Kumar et al., Current Cancer Drug Targets 2017, 17 (4), 357-375, and is represented by the structural formula indicated below:

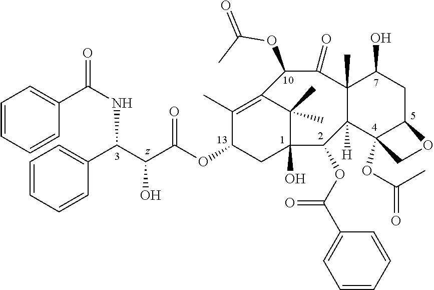

In some embodiments, the taxane is cabazitaxel. Cabazitaxel (Jevtana™) is described in Ojima et al., Expert Opin. Ther. Patents 2016, 26 (1), 1-10 20, and B. Kumar et al., Current Cancer Drug Targets 2017, 17 (4), 357-375, and is represented by the structural formula indicated below:

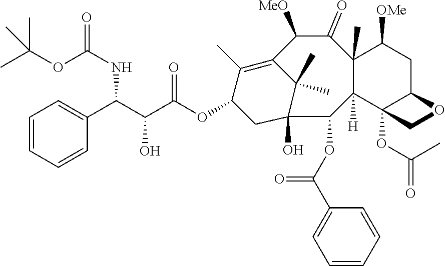

In some embodiments, the taxane is larotaxel. Larotaxel is described in Ojima et al., Expert Opin. Ther. Patents 2016, 26 (1), 1-20, and B. Kumar et al., Current Cancer Drug Targets 2017, 17 (4), 357-375, and is represented by the structural formula indicated below:

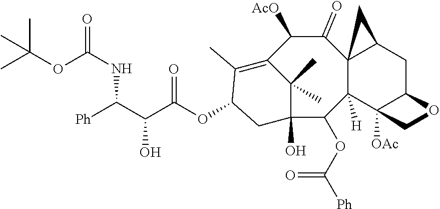

In some embodiments, the taxane is ortataxel. Ortataxel is described in Ojima et al., Expert Opin. Ther. Patents 2016, 26 (1), 1-20, B. Kumar et al., Current Cancer Drug Targets 2017, 17 (4), 357-375, and I. Ojima et al. J. Nat. Prod. 2018, 81, 703-721, and is represented by the structural formula indicated below:

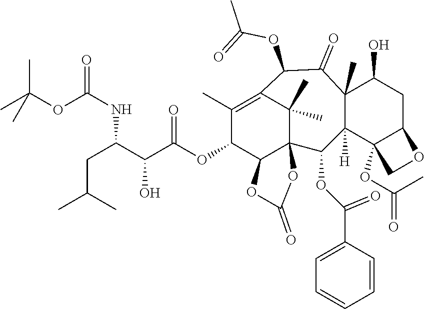

In some embodiments, the taxane is tesetaxel. Tesetaxel is described in Ojima et al., Expert Opin. Ther. Patents 2016, 26 (1), 1-20, B. Kumar 10 et al., Current Cancer Drug Targets 2017, 17 (4), 357-375, and M. Shionoya et al., Cancer Sci 2003, 94 (5), 459-466, and is represented by the structural formula indicated below:

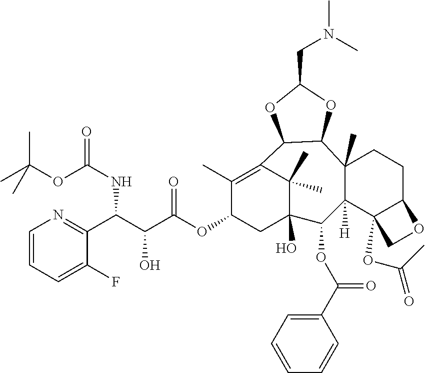

In some embodiments, the taxane is milataxel. Milataxel is described in Ojima et al., Expert Opin. Ther. Patents 2016, 26 (1), 1-20, D. Sampath et al., Mol Cancer Ther 2003, 2, 873-884, and R. K. Ramanathan et al., Cancer Chemother Pharmacol 2008, 61, 453-458, and is represented by the structural formula indicated below:

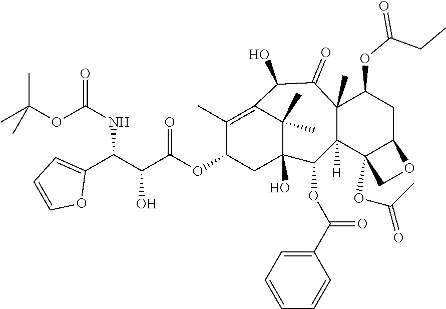

In some embodiments, the taxane is docosahexaenoic acid (DHA)-paclitaxel. Docosahexaenoic acid (DHA)-paclitaxel (Taxoprexin™) is described in Ojima et al., Expert Opin. Ther. Patents 2016, 26 (1), 1-20, and I. Ojima et al., Future Med. Chem. 2012, 4 (1), 33-50, and is represented by the structural formula indicated below:

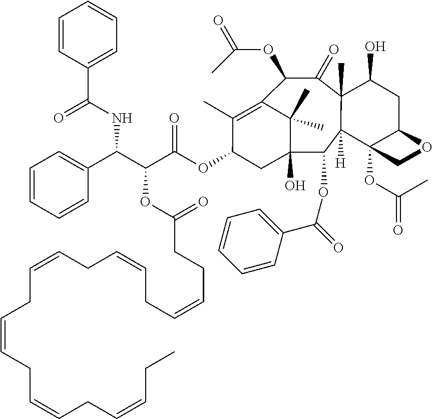

In some embodiments, the taxane is poly(L-glutamic acid) PG-paclitaxel. Poly(L-glutamic acid) PG-paclitaxel (Opaxio™) is described in Ojima et al., Expert Opin. Ther. Patents 2016, 26 (1), 1-20 and J. W. Singer et al. in Macromolecular Anticancer Therapeutics, Cancer Drug Discovery and Development, L. H. Reddy, P. Couvreur (editors), Springer New York Dordrecht Heidelberg London, 2010, chapter 4, page 133-161, and is represented by the structural formula indicated below, wherein y, a, and b are independent variables:

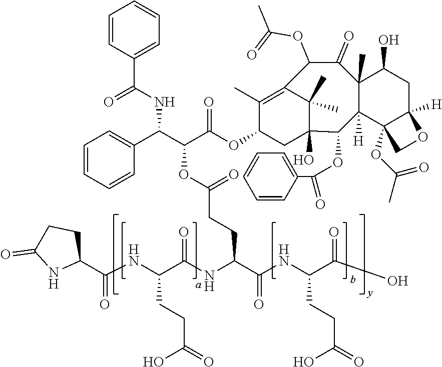

In some embodiments, the taxane is BMS-184476 (7-O-methylthiomethyl paclitaxel). BMS-184476 (7-O-methylthiomethyl paclitaxel) is described in Ojima et al., Expert Opin. Ther. Patents 2016, 26 (1), 1-20, and T. J. Altstadt et al., J. Med. Chem. 2001, 44, 4577-4583, and is represented by the structural formula indicated below:

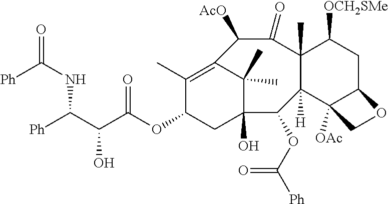

In some embodiments, the taxane is SB-T-1214. SB-T-1214 is described in Ojima et al., Expert Opin. Ther. Patents 2016, 26 (1), 1-20, I. Gut et al., Xenobiotica 2006, 36 (9), 772-792, and I. Ojima et al., J. Med. Chem. 2008, 51, 3203-3221, and is represented by the structural formula indicated below:

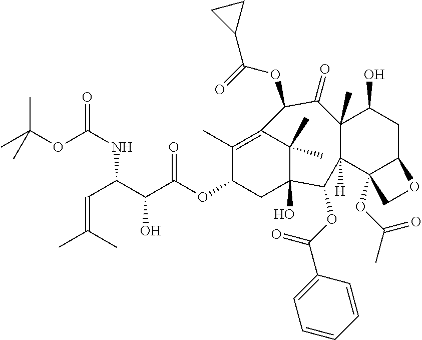

In some embodiments, the taxane is SB-T-1216. In some embodiments, the taxane is SB-T-121602. SB-T-1216 and SB-T-121602 are described in Ojima et al., Expert Opin. Ther. Patents 2016, 26 (1), 1-20, I. Gut et al., Xenobiotica 2006, 36 (9), 772-792, and I. Ojima et al. J. Nat. Prod. 2018, 81, 703-721, and is represented by the structural formula indicated below:

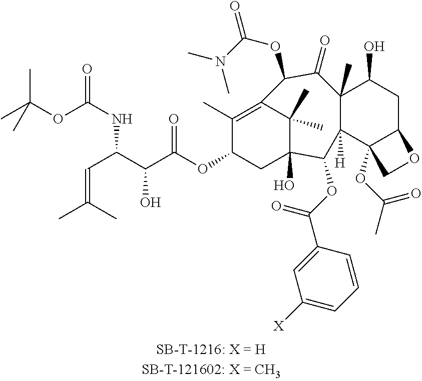

In some embodiments, the taxanc is SB-T-12854. SB-T-12854 is described in Ojima ct al., Expert Opin. Ther. Patents 2016, 26 (1), 1-20, and I. Ojima et al. J. Nat. Prod. 2018, 81, 703-721, and is represented by the structural formula indicated below:

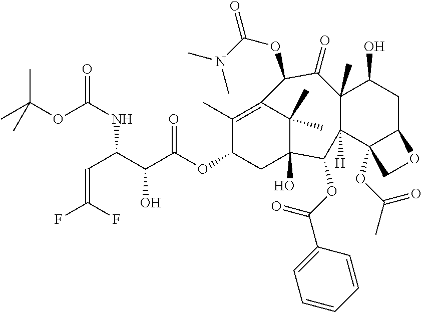

In some embodiments, the taxane is DHA-SB-T-1214. DHA-SB-T-1214 is described in Ojima et al., Expert Opin. Ther. Patents 2016, 26 (1), 1-20, and is represented by the structural formula indicated below:

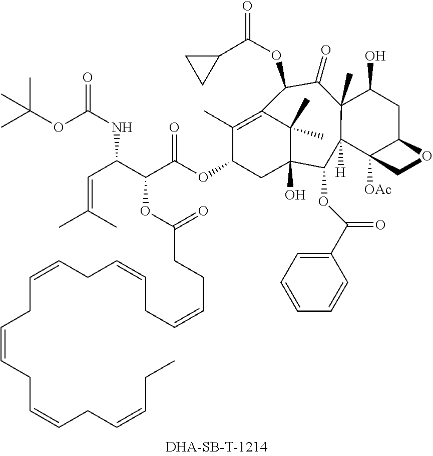

In some embodiments, the taxane is abeo-taxane 15a.2. Abeo-taxane 15a.2 is described in Ojima et al., Expert Opin. Ther. Patents 2016, 26 (1), 1-20, and WO 2013/106029 A1, and is represented by the structural formula indicated below:

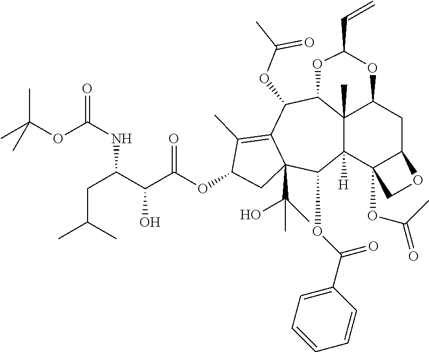

In some embodiments, the taxane is cabazitaxel-7,10-d6. Cabazitaxel-7,10-d6 is described in Ojima et al., Expert Opin. Ther. Patents 2016, 26 (1), 1-20, 5 and is represented by the structural formula indicated below:

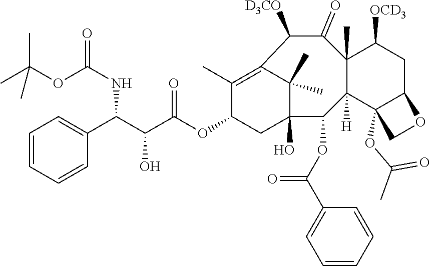

In some embodiments, the taxane is docetaxel-f3-t-Boc. Docetaxel-f3-t-Boc is described in Ojima et al., Expert Opin. Ther. Patents 2016, 26 (1), 1-20, and is represented by the structural formula indicated below:

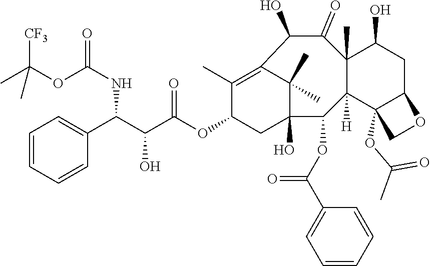

In some embodiments, the taxane is docetaxel-d9-t-Boc. Docetaxel-d9-t-Boc is described in Ojima et al., Expert Opin. Ther. Patents 2016, 26 (1), 1-20, and is represented by the structural formula indicated below:

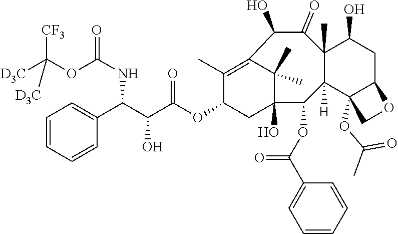

In some embodiments, the taxane is ANG-1005. ANG-1005 is described in Ojima et al., Expert Opin. Ther. Patents 2016, 26 (1), 1-20, and 5 WO 2010/121379 A1, and is represented by the structural formula indicated below:

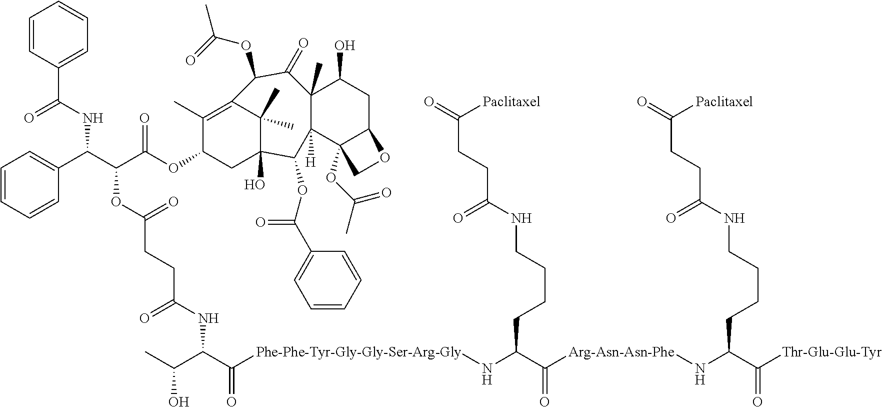

In some embodiments, the taxane is cobalamin-paclitaxel. Cobalamin-paclitaxel is described in Ojima et al., Expert Opin. Ther. Patents 2016, 26 (1), 1-20, and WO 2008/115805 A2, and is represented by the structural formula indicated below:

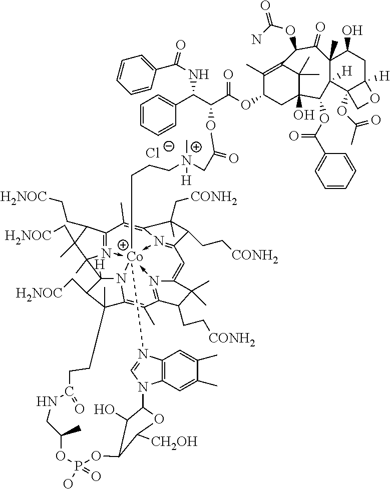

In some embodiments, the taxanc is FK506-PEG3-docetaxel. FK506-PEG3-docetaxel is described in Ojima et al., Expert Opin. Ther. Patents 2016, 26 (1), 1-20, and WO 2011/130317 A2, and is represented by the structural formula indicated below:

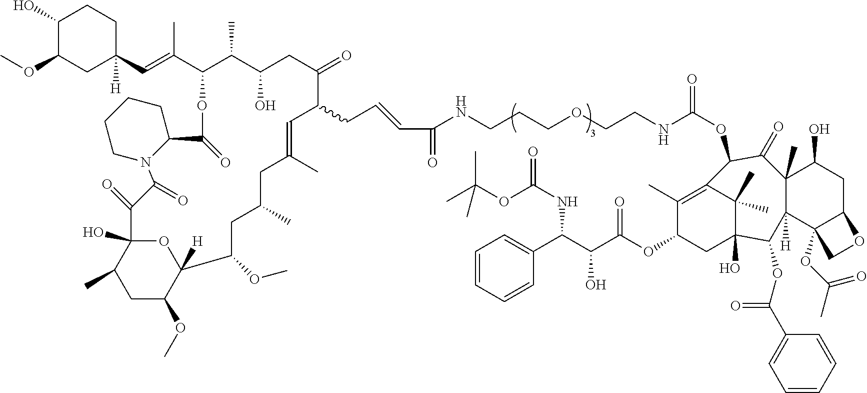

In some embodiments, the taxane is biotin-docetaxel (IDD-1010). Biotin-docetaxel (IDD-1010) is described in Ojima et al., Expert Opin. Ther. Patents 2016, 26 5 (1), 1-20, and WO 2014/191989 A1, is represented by the structural formula indicated below:

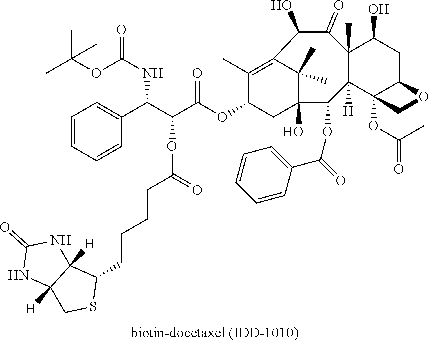

In some embodiments, the taxane is biotin-SB-T-1214 (BLT-1). Biotin-SB-T-1214 (BLT-1) is described in Ojima et al., Expert Opin. Ther. Patents 2016, 26 (1), 1-20, and is represented by the structural formula indicated below:

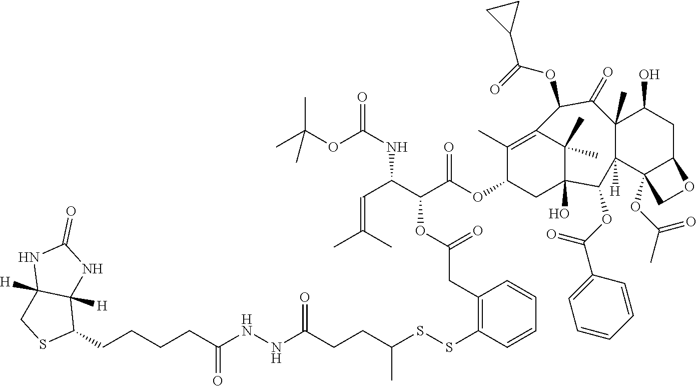

In some embodiments, the taxane is 4-ARM-PEG20K-CM-Gly-d9-DOC. 4-ARM-PEG20K-CM-Gly-d9-DOC is described in Ojima et al., Expert Opin. Ther. Patents 2016, 5 26 (1), 1-20, and WO 2012/088422 A1, and is represented by the structural formula indicated 5 bclow:

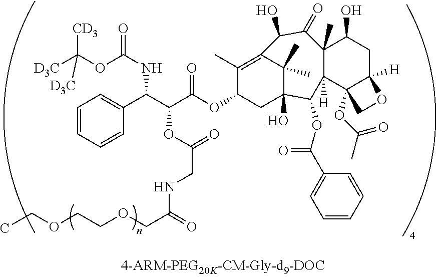

In some embodiments, the taxane is 4-ARM-PEG20K-BA-d9-DOC. 4-ARM-PEG20K-BA-d9-DOC is described in Ojima et al., Expert Opin. Ther. Patents 2016, 26 (1), 1-20, and WO 2012/088422 A1, and is represented by the structural formula indicated below:

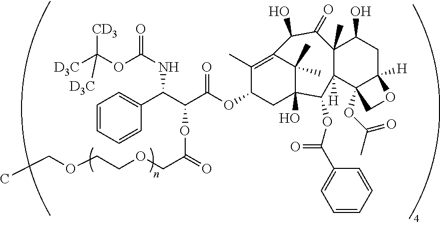

The abbreviations listed below have been used for certain substituents within the structures presented above: Me for methyl-, Ac for acetyl-, Ph for phenyl and Bz for benzoyl-.

In some embodiments, the taxane is liposomal paclitaxel. The term “liposomal paclitaxel” as used herein refer to liposomal formulations of paclitaxel. Exemplary liposomal formulations of paclitaxel include Lipusu™, a liposomal paclitaxel formulation developed by Sike Pharmaceutical Co. Ltd., Nanjing, Jiangsu, P. R. China, which has been approved by the State Food and Drug Administration of China. Exemplary liposomal formulations of paclitaxel are described in S. Koudelka, J. Turanek, Journal of Controlled Release 2012, 163, 322-334.

In some embodiments, the taxane is Nab™-paclitaxel (ABI-007; Abraxane™). Nab™-paclitaxel is a nanoparticle albumin-bound paclitaxel. The human albumin-stabilized paclitaxel particles have an average size of ˜130 nm as described Ojima et al., Expert Opin. Ther. Patents 2016, 26 (1), 1-20.

Additional nano-based drug delivery systems for paclitaxel are Genexol™ PM and Taclantis™ (Bevetex™) as described in Pi-Ling Cou et al., International Journal of Nanomedicine 2020, 15, 1731-1743). Genexol™ PM comprises polymeric micelle paclitaxel having an average 27 particle size between 25 and 50 nm. The micelles comprise a monomethoxy poly(ethylene glycol)-block-poly(D,L-lactide) (mPEG-PDLLA) copolymer. Taclantis™ (Bevetex™) is a paclitaxel injection concentrate for nanodispersion and based on a polyvinylpyrrolidone/paclitaxel self-assembly. The formulation of Taclantis™ (Bevetex™) is cremophor free and human serum albumin free. The average particle size is between 100 and 110 nm.

The methods described herein may be used to treat any type of cancer. In some embodiments, the cancer is bone cancer. For example, the caner may be a bone cancer such as osteosarcoma, chondrosarcoma, or Ewing sarcoma. In some embodiments, the cancer is a cancer which has metastasized to the bone of the subject. For example, the cancer which has metastasized to the bone of the subject may be kidney cancer, lung cancer, lymphoma, multiple myeloma, prostate cancer, or thyroid cancer. In some embodiments, the cancer which has metastasized to the bone of the subject is prostate cancer.

Treating cancer (e.g. prostate cancer) with bone metastasis may involve, at least in part, reducing the overall tumor burden in the bone of the afflicted subject. For example, treating cancer with bone metastasis may involve reducing the size and/or number of tumors in the bones of the afflicted subject. In addition, treating the cancer with bone metastasis may involve reducing the size and/or number of tumors elsewhere in the subject (e.g. at the site of origin of the cancer, such as at the prostate).

In some embodiments, provided herein are methods of treating a tumor in the bone of a subject. The method comprises providing to the subject a CXCR4 inhibitor and a chemotherapeutic agent, as described herein. The tumor may be the result of bone cancer, such osteosarcoma, chondrosarcoma, or Ewing sarcoma. In some embodiments, the tumor is a result of bone metastasis of any cancer. In some embodiments, the tumor is the result of bone metastasis of a cancer selected from the group consisting of kidney cancer, lung cancer, lymphoma, multiple myeloma, prostate cancer, and thyroid cancer. In some embodiments, the tumor is the result of bone metastasis of prostate cancer.

Any suitable amount of the CXCR4 inhibitor may be used. In some embodiments, the dose of the CXCR4 inhibitor is about 0.1 mg inhibitor/kg body weight to about 20 mg/kg.

The CXCR4 inhibitor may be administered by any suitable route. In some embodiments, the CXCR4 inhibitor is administered orally. In some embodiments, the CXCR4 inhibitor is formulated as a liquid containing one or more suitable carriers for parenteral administration (e.g. injection). For example, the CXCR4 inhibitor may be formulated as a liquid for subcutaneous, intravenous, intramuscular, or intrathecal injection.

Any suitable administration schedule for the CXCR4 inhibitor may be used. The CXCR4 inhibitor may be administered once or multiple times. For example, the CXCR4 inhibitor may be administered to the subject once. As another example, the CXCR4 inhibitor may be administered to the subject multiple times. Administration of the CXCR4 inhibitor may occur multiple times in a single day, or multiple times over the course of different days. Administration may continue until the cancer is treated. For example, administration may continue until the tumor burden (e.g. size of a tumor and/or number of tumors) in the subject is reduced.

Any suitable amount of the chemotherapeutic agent may be used. In some embodiments, the dose of the chemotherapeutic agent is about 0.1 mg inhibitor/kg body weight to about 20 mg/kg.

The chemotherapeutic agent may be administered by any suitable route. In some embodiments, the chemotherapeutic agent is administered orally. In some embodiments, the chemotherapeutic agent is formulated as a liquid containing one or more suitable carriers for parenteral administration (e.g. injection). For example, the CXCR4 inhibitor may be formulated as a liquid for subcutaneous, intravenous, intramuscular, or intrathecal injection. Any suitable administration schedule for the chemotherapeutic agent may be used. The chemotherapeutic agent may be administered once or multiple times. For example, the chemotherapeutic agent may be administered to the subject once. As another example, the chemotherapeutic agent may be administered to the subject multiple times. Administration of the chemotherapeutic agent may occur multiple times in a single day, or multiple times over the course of different days. For example, administration may occur daily, every two days, every three days, every four days, every five days, every six days, every seven days, every eight days, every nine days, every 10 days, every 11 days, every 12 days, ever 13 days, every 2 weeks, every 3 weeks, or monthly. Administration may continue until the cancer is treated. For example, administration may continue until the tumor burden (e.g. size of a tumor and/or number of tumors) in the subject is reduced.

In some embodiments the CXCR4 inhibitor and/or the chemotherapeutic agent may be formulated for oral administration. For oral administration, the agent (e.g. the CXCR4 inhibitor, the chemotherapeutic agent) or a salt thereof may be combined with one or more carriers to be formulated as tablets, pills, dragees, capsules, liquids, gels, syrups, slurries, suspensions etc., for oral ingestion by a patient to be treated.

As described above, in some embodiments the CXCR4 inhibitor and/or the chemotherapeutic agent may be formulated for injection.

In some aspects, provided herein are compositions. In some embodiments, provided herein are compositions comprising a CXCR4 inhibitor as described herein and a taxane as described herein. Any suitable CXCR4 inhibitor may be used, including, for example, antibodies, antibody fragments, aptamers, peptides, and small molecules. In some embodiments, the CXCR4 inhibitor is a peptide. In some embodiments, the CXCR4 inhibitor is the peptide Balixafortide (POL6326). In some embodiments, the CXCR4 inhibitor is Plerixafor (AMD3100). In some embodiments, the CXCR4 antagonist is BL-8040 (previously called BKT140).

In some embodiments, the CXCR4 inhibitor is Balixafortide or an analogue thereof. Balixafortide and analogues thereof are described above. Suitable taxanes are described above and include, for example, paclitaxel, docetaxel, abraxane, taxotere, cabazitaxel, or analogues thereof. In some embodiments, the taxane is selected from the group consisting of paclitaxel, docetaxel, abraxane, taxotere and cabazitaxel. In some embodiments, the taxane is docetaxel or an analogue thereof, as described above.

The compositions described herein may be used to treat cancer in a subject. The compositions may be used to treat any type of cancer. In some embodiments, the cancer is bone cancer. For example, the cancer may be a bone cancer such as osteosarcoma, chondrosarcoma, or Ewing sarcoma. In some embodiments, the cancer is a cancer which has metastasized to the bone of the subject. For example, the cancer which has metastasized to the bone of the subject may be kidney cancer, lung cancer, lymphoma, multiple myeloma, prostate cancer, or thyroid cancer. In some embodiments, the cancer which has metastasized to the bone of the subject is prostate cancer.

### EXAMPLES

### Example 1

Experiments were performed to investigate the efficacy of the CXCR4 inhibitor Balixafortide as an inhibitor of metastatic prostate cancer.

Methods: 59 severe combined immunodeficiency (SCID) mice received intratibial injection with PC3-luc cells (2×10⁴ cells in 20 ul). Balixafortide (20 mg/kg) (BAL) and vehicle were injected subcutaneously twice daily starting on day one and Docetaxel (5 mg/kg) (DOC) was injected intraperitoneal once a week starting on day one. The tumor growth in mouse tibia was checked weekly by BLI.

After 30 days, at the time of necropsy, tumor growth in mouse tibia was checked by radiograph and mice were sacrificed, and serum and bone were collected. Serum was kept at −80° C. and tibia were stored in 10% buffered formalin at room temperature for 48 hours before being stored at 4° C.

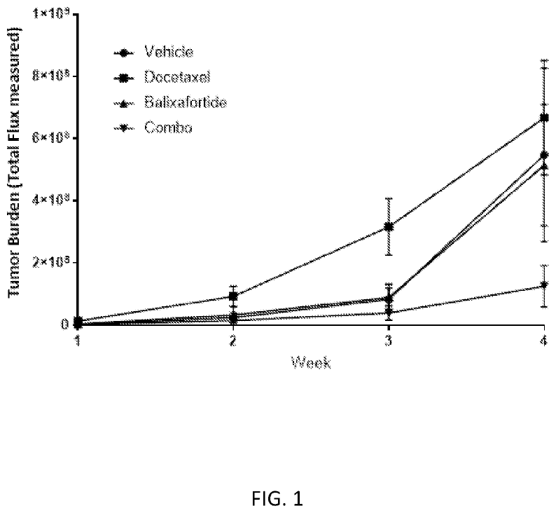

Results: In each group, mice that never developed tumor were removed from the analysis. While neither BAL nor DOC impacted tumor growth as sole agents, the combination of BAL and DOC decreased the total tumor burden significantly (p=0.009) (FIG. 1). In addition, the combination of BAL and DOC at week 4 has a lower tumor burden than the other treatments.

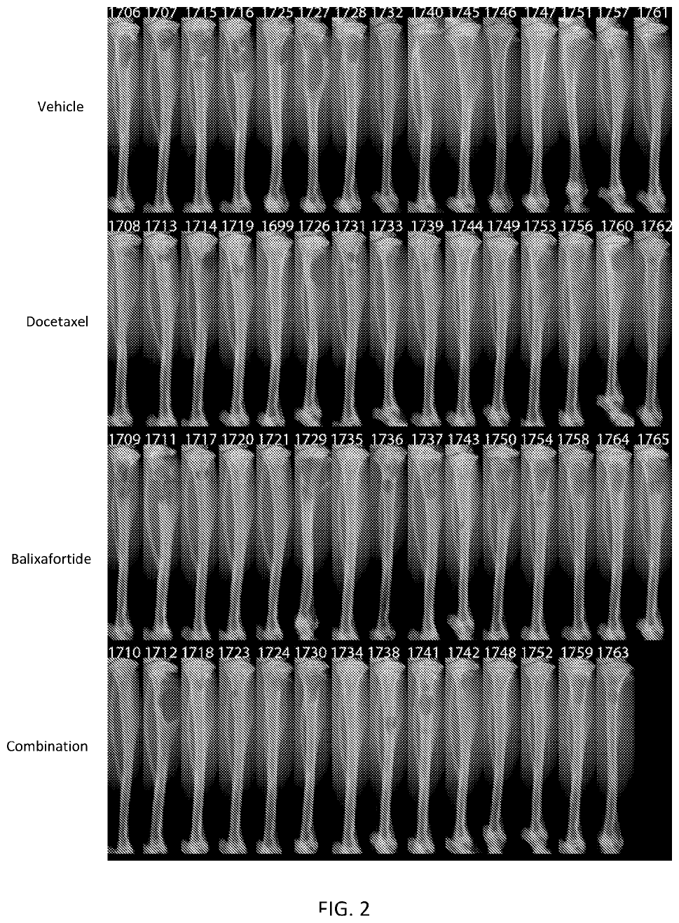

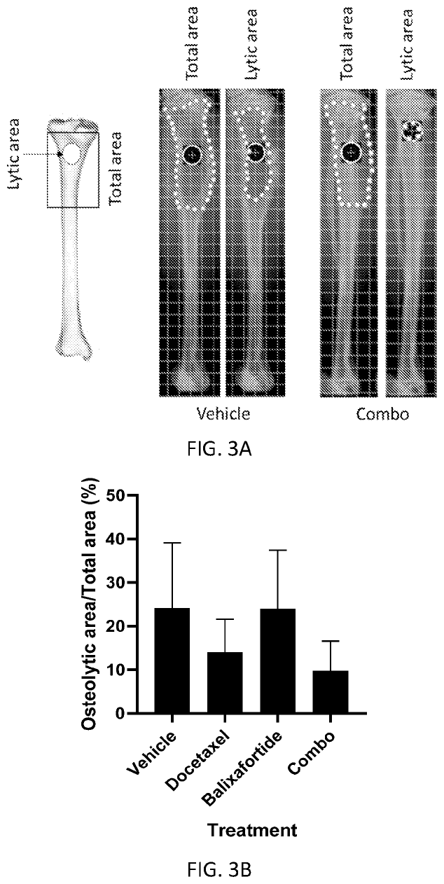

Radiograph imaging of the tibia on the day of necropsy show that animals that received a combination of BAL and DOC show less overall bone degradation from the PC3 cells (FIG. 2). Some animals were removed from analysis as they did not appear to show any signal from bioluminescence imaging (BLI). Radiographic images were imported into SketchandCalc software. The proximal third of the tibia was traced to calculate its area and defined as total area, then osteolytic areas were traced to calculate the total osteolytic area (FIG. 3A) and the percent osteolytic area/bone area was calculated. Due to the variance in osteolytic areas, there were no significant differences among the groups; however, the mean of the combination appears lower than docetaxel alone, which is lower than vehicle or Balixafortide (FIG. 3B)

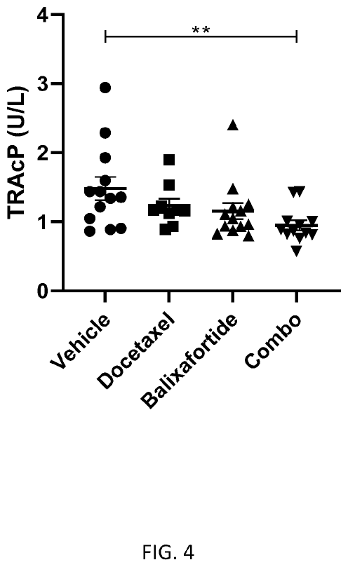

Serum was analyzed for TRAcP levels, an indicator of bone resorption. Animals that received a combination of BAL and DOC demonstrate lower TRAcP levels than the vehicle group (p=0.01) as shown in FIG. 4, Table 1, and Table 2. All samples were averaged by taking two or three technical replicates based on how much serum was collected at the time of nccropsy. Data tables included to show statistical significance.

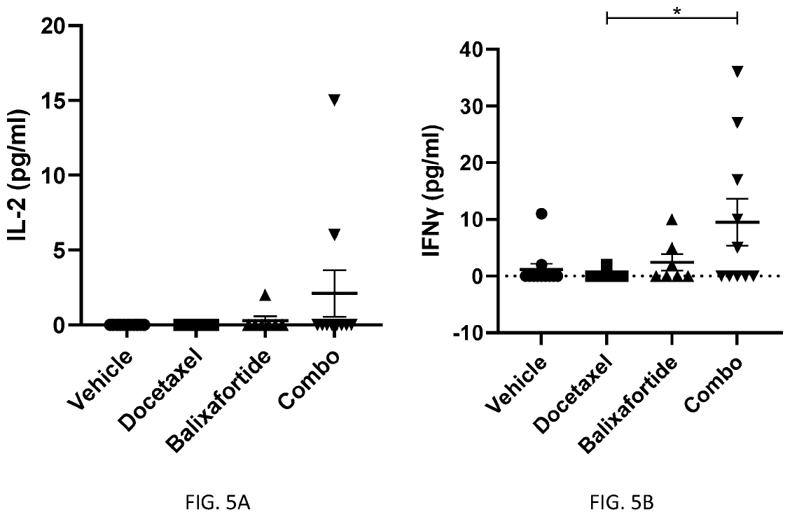

Serum was analyzed for interleukin-2 (IL-2) and IFN-gamma (IFN-γ) levels using enzyme-linked immunoassay (ELISA). Animals that received a combination of BAL and DOC had a trend towards elevated IL-2 and IFN-γ serum levels compared to the other groups (FIG. 5).

TABLE 1

| | | | | | |
|---|---|---|---|---|---|
| | Mean Diff. | | | | |
| Dunnett's multiple | 95.00% | | | Adjusted | |
| comparisons test | CI of diff. | Significant? | Summary | P Value | A-? |

Docetaxel 5.096 value included

| | | | | | |
|---|---|---|---|---|---|
| Vehicle vs. Combo | 0.5312-0.1493 | No | ns | 0.1571D | Combo |
| | to 1.212 | | | | |

Docetaxel 5.096 value removed (outlier)

| | | | | | |
|---|---|---|---|---|---|
| Vehicle vs. Combo | 0.53120.1106 | Yes | ** | 0.01D | Combo |
| | to 0.9518 | | | | |

TABLE 2

| Unpaired t test | |
|---|---|
| P value | 0.0096 |
| P value summary | ** |
| Significantly different (P < 0.05)? | Yes |
| One- or two-tailed P value? | Two-tailed |
| t, df | t = 2.826, df = 23 |

Taken together, the data show that the combination of DOC and BAL inhibits intraosseous growth of PC-3 prostate cancer cells compared to either drug alone.

It is understood that the foregoing detailed description and accompanying examples arc merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

Any patents and publications referenced herein are herein incorporated by reference in their entireties.

1. A method of treating cancer in a subject, comprising providing to the subject a CXCR4 inhibitor and a taxane, wherein the cancer is a bone cancer or wherein the cancer has metastasized to the bone of a subject.

2. The method of claim 1, wherein the bone cancer is osteosarcoma, chondrosarcoma, or Ewing sarcoma, or wherein the cancer which has metastasized to the bone of a subject is selected from the group consisting of kidney cancer, lung cancer, lymphoma, multiple myeloma, prostate cancer and thyroid cancer.

3. The method of claim 2, wherein the cancer which has metastasized to the bone of a subject is prostate cancer.

4. The method of claim 1, wherein the CXCR4 inhibitor is a peptide.

5. The method of claim 1, wherein the CXCR4 inhibitor is Balixafortide.

6. The method of claim 1, wherein the taxane is selected from the group consisting of paclitaxel, docetaxel, abraxane, taxotere and cabazitaxel.

7. A method of treating a tumor in a bone of a subject, comprising providing to the subject a CXCR4 inhibitor and a taxane.

8. The method of claim 7, wherein the CXCR4 inhibitor is a peptide.

9. The method of claim 7, wherein the CXCR4 inhibitor is Balixafortide.

10. The method of claim 7, wherein the taxane is selected from the group consisting of paclitaxel, docetaxel, abraxane, taxotere and cabazitaxel.

11. The method of claim 7, wherein the tumor is the result of bone metastasis of a cancer selected from the group consisting of kidney cancer, lung cancer, lymphoma, multiple myeloma, prostate cancer and thyroid cancer.

12. The method of claim 7, wherein the tumor is the result of bone metastasis of prostate cancer.

13. A composition comprising a CXCR4 inhibitor and a taxane for use in a method of treating cancer in a subject, wherein the cancer is a bone cancer or wherein the cancer has metastasized to the bone of a subject.

14. The composition of claim 13, wherein the CXCR4 inhibitor is a peptide.

15. The composition of claim 14, wherein the CXCR4 inhibitor is Balixafortide.

16. The composition of claim 13, wherein the taxane is selected from the group consisting of paclitaxel, docetaxel, abraxane, taxotere and cabazitaxel.

17. The composition of claim 13, wherein the bone cancer is osteosarcoma, chondrosarcoma, or Ewing sarcoma, or wherein the cancer which has metastasized to the bone of a subject is selected from the group consisting of kidney cancer, lung cancer, lymphoma, multiple myeloma, prostate cancer and thyroid cancer.
